(19) United States
(12) Patent Application Publication
(10) Pub. No.: US 2024/0398342 A1
(43) Pub. Date: Dec. 5, 2024

(54) NONINVASIVE SYSTEM AND METHOD FOR MEASURING HYPOGLYCEMIA AND HYPERGLYCEMIA

(71) Applicant: Becton, Dickinson and Company, Franklin Lakes, NJ (US)

(72) Inventors: Eric Ray SUN, Waltham, MA (US); J. Richard GYORY, Sudbury, MA (US)

(73) Assignee: Becton, Dickinson and Company, Franklin Lakes, NJ (US)

(21) Appl. No.: 18/690,584

(22) PCT Filed: Sep. 9, 2022
(86) PCT No.: PCT/US2022/076234

§ 371 Date: Mar. 8, 2024

Related U.S. Application Data
(60) Provisional application No. 63/242,687, filed on Sep. 10, 2021.

(51) Int. Cl. A61B 5/00; A61B 5/0205; A61B 5/0531; A61B 5/145; A61B 5/332

(52) U.S. Cl. CPC A61B 5/4866; A61B 5/002; A61B 5/02055; A61B 5/681; A61B 5/7282; A61B 5/746; A61B 5/0531; A61B 5/14542; A61B 5/332; A61B 5/6815

(57) ABSTRACT

A wearable unit is provided including a plurality of sensors including a temperature sensor configured to sense a temperature of a patient, a pulse oximeter configured to sense an oxygen saturation of the patient, an accelerometer configured to sense a breathing pattern of the patient, a skin conductance sensor configured to sense a skin conductance of the patient, and an electrocardiogram (ECG) monitor configured to sense an ECG pattern of the patient. The wearable unit may be configured to be worn on the patient's wrist or may be configured to be worn in the patient's ear. Information output by the sensors may be used to determine whether the patient is hyperglycemic or hypoglycemic and to provide an alert the patient or patient's caregiver. Information from the wearable unit may be wirelessly transmitted to an external device.

### BACKGROUND

### 1. Field

Apparatuses and methods consistent with exemplary embodiments relate to systems and methods for noninvasive measurement and detection of hypoglycemia and hyperglycemia, and more specifically to such systems and methods utilizing physiological parameters without the use of blood glucose.

### 2. Description of the Related Art

Diabetes is a group of diseases characterized by high levels of blood glucose resulting from the inability of diabetic patients to maintain proper levels of insulin production when required. Complications from diabetes can be minimized by utilizing one or more treatment options. The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient, and requires a continuing measurement and detection of the hypoglycemic or hyperglycemic state of a patient.

Hypoglycemia, in lay terms known as “low blood sugar” or “insulin shock”, is an undesirable and potentially lethal side-effect of insulin treatment in diabetes mellitus. Hypoglycemia triggers a hypothalamic stress response, resulting in increased activity in the sympathetic nervous system and release of the catecholamine hormones epinephrine and norepinephrine from the adrenal medulla. Catecholamine release into the blood stream induces excitatory or adrenergic responses such as shakiness, increased heart rate and perspiration, and cutaneous vasoconstriction, potentially resulting in paleness and a drop in skin temperature. Over a period of hours, declining blood glucose concentration may ultimately affect the brain and lead to neuroglycopenic symptoms such as dizziness, impaired coordination, mental confusion, and altered behavior. If left untreated, extreme hypoglycemia may result in coma, brain damage or death.

Upon becoming aware of early autonomic indicators like increased perspiration or heart palpitations, a diabetic patient can correct mild hypoglycemia by taking a fast-acting carbohydrate, such as glucose tablets, fruit juice, or candies. However, awareness of adrenergic symptoms may be compromised by diabetic autonomic neuropathy, a nervous disorder that is likely attributable to a combination of factors including high blood glucose and a long duration of diabetes.

Awareness of physical symptoms is also reduced or inhibited by “hypoglycemia unawareness”, an increased tolerance to low blood sugar which develops as a result of repeated hypoglycemic episodes. Since epinephrine response is blunted during sleep and as a consequence of hypoglycemia unawareness caused by neuropathy or frequent lows, a sleeping diabetic patient may not awaken until after nueroglycopenic symptoms are established, in which case the patient in a confused mental state may neglect or even resist treatment. Therefore, it is particularly important to provide methods of preventing nocturnal hypoglycemic events at the earliest possible stage of detection, so that development of hypoglycemia unawareness is avoided.

Hyperglycemia, also known as “high blood sugar,” is a condition in which an excessive amount of glucose circulates in the blood plasma. Acute hyperglycemia, involving glucose levels that are extremely high, is a medical emergency and can rapidly produce serious complications such as fluid loss through osmotic diuresis. Such hyperglycemia may be caused by low insulin levels, such as in diabetes mellitus, type 1, and/or by resistance to insulin at the cellular level, such as in diabetes mellitus, type 2. Ketoacidosis may be the first symptom of immune-mediated diabetes, particularly in children and adolescents. Also, patients with immune-mediated diabetes, can change from modest fasting hyperglycemia to severe hyperglycemia and even ketoacidosis as a result of stress or an infection. Thus, it is equally important to provide methods of preventing nocturnal hyperglycemic events.

Diabetic patients have few options of detecting hypoglycemia or hyperglycemia while they are sleeping. Existing options generally rely on detection via skin conductivity and body temperature, which may be used to detect hypoglycemia, but which are not sufficient for detection of hyperglycemia. Alternative options require the use of an implantable sensor which may last only 10 days. Less expensive, and more versatile options are needed, in particular, for patients with diabetes mellitus, type 2, who are often particularly cost-sensitive.

### SUMMARY

Example embodiments may address at least the above problems and/or disadvantages and other disadvantages not described above. Also, example embodiments are not required to overcome the disadvantages described above, and may not overcome any of the problems described above.

One or more example embodiments may provide a wearable unit comprising a housing configured to be worn on a body of a patient; and an electronics module. The electronics module may comprise a power source, a plurality of sensors comprising a temperature sensor configured to sense a temperature of the patient, a pulse oximeter configured to sense an oxygen saturation of the patient, an accelerometer configured to sense a breathing pattern of the patient, a skin conductance sensor configured to sense a skin conductance of the patient, and an electrocardiogram (ECG) monitor configured to sense an ECG pattern of the patient. The electronic module may further comprise a microcontroller configured to receive an output from one or more of the plurality of sensors and to determine, whether the output indicates one of hyperglycemia and hypoglycemia.

The wearable unit may further comprise a wristband configured to attach the housing to a wrist of the patient or an earpiece configured to hold the housing within an ear of the patient.

The microcontroller may further comprising a Bluetooth module; and the microcontroller may be further configured to transmit information to an external device, the information comprising at least one of the output from the one or more of the plurality of sensors and a determination of whether the output indicates one of hyperglycemia and hypoglycemia.

The electronics module may further comprise an output unit, and the microcontroller may be configured to output at least one of a visible alert and an audible alert to the patient based on whether the output indicates one of hyperglycemia and hypoglycemia.

One or more example embodiments may provide a sensing system comprising an external device; and the wearable unit. The external device may be configured to receive the information from the wearable device and to determine whether the output indicates one of hyperglycemia and hypoglycemia.

The external device may be further configured to determine to output at least one of a visible alert and an audible alert to the patient based on whether the output indicates one of hyperglycemia and hypoglycemia.

One or more example embodiments may provide a sensing method comprising: a wearable unit sensing a temperature of a patient wearing the wearable unit, an oxygen saturation of the patient, a breathing pattern of the patient, a skin conductance of the patient, and an electrocardiogram (ECG) pattern of the patient; transmitting, from the wearable unit to an external device, the oxygen saturation, the breathing pattern, the skin conductance, and the electrocardiogram (ECG) pattern; the external device determining whether the output indicates one of hyperglycemia and hypoglycemia; and the external device outputting at least one of a visible alert and an audible alert based on whether the output indicates one of hyperglycemia and hypoglycemia.

### BRIEF DESCRIPTION OF THE DRAWINGS

The above and/or other example aspects will become apparent and more readily appreciated from the following description of example embodiments, taken in conjunction with the accompanying drawings in which:

FIG. 1 is a wrist-wearable unit according to an example embodiment;

FIG. 2 is a schematic illustration of an electronics module of a wearable module according to an example embodiment;

FIG. 3 is a schematic illustration of sensors of a wearable unit according to an example embodiment;

FIG. 4 is an in-ear wearable unit according to an example embodiment;

FIG. 5 illustrates a system according to an example embodiment;

FIG. 6 is a flow chart of an operation of a wearable unit according to an example embodiment; and

FIG. 7 is a flow chart of operations of a wearable unit and an external device according to an example embodiment.

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including,” “comprise,” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names—this document does not intend to distinguish between components that differ in name but not function.

Matters of these example embodiments that are obvious to those of ordinary skill in the technical field to which these example embodiments pertain may not be described here in detail.

One or more example embodiments describe a system and method for noninvasive detection of hypoglycemia and hyperglycemia.

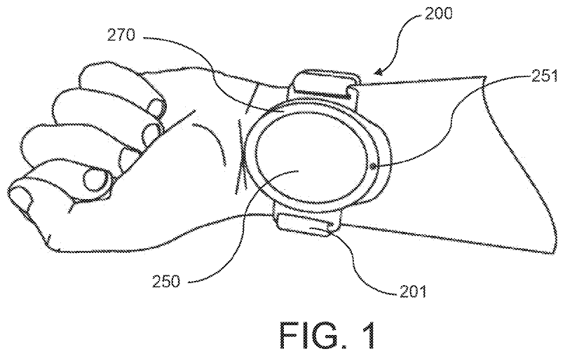

According to an example embodiment, as shown in FIG. 1, a system includes a wrist-wearable unit 200, such as a watch-like unit, for measuring one or more physiological symptoms via detection on a patient's wrist. The wearable unit 200 may include a wrist strap 201 which may be elastomeric, fabric, silicon, or another material, and may be secured to a patient's wrist via its elastic tension alone or may include a fastener (not shown). The wearable unit 200 further includes the electronics module 250 to be secured by the strap to the patient's wrist. The wearable unit 200 may further include an actuator 251 configured to enable the patient to activate or deactivate the physiological monitoring of the electronics module 250. Of course, the actuator 251 may alternately be incorporated into the electronics module 250 itself.

The electronics module 250 includes a housing 270, which may include one or more housing members connected to each other, as would be understood by one of skill in the art. Example housing members may be injection-molded from high-impact plastic and glued together or held together by screws or another means as would be understood by one of skill in the art. The housing 270 may be water-tight.

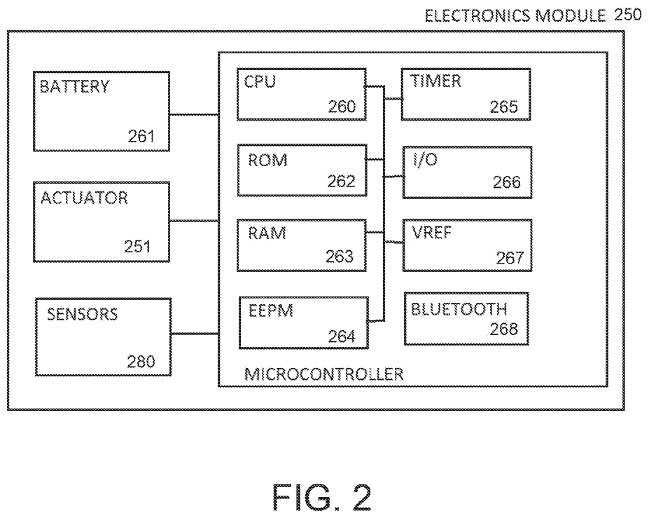

FIG. 2 illustrates a schematic diagram of the electronics module 250 in accordance with an example embodiment. Mounted within the housing 270 is a microcontroller 260, provided at least for operating and controlling the electronics module 250, and a battery 261 and actuator 251 connected to the microcontroller. The microcontroller 260 may be a CMOS integrated circuit comprising the functional elements of a central processing unit (CPU) 260, a read-only memory (ROM) 262, a random access memory (RAM) 263, an electrically-erasable programmable memory (EEPM) 264, a timer 265, one or more input/output ports 266, and a programmable voltage reference (VREF) 267.

The CPU 260 executes instructions according to a program stored in the ROM 262. The RAM X263X provides the CPU 260 with means for temporary data storage. The EEPM 264 provides the CPU 260 with means for non-volatile data storage. The input/outputs 266 allow the CPU 260 to receive and output signals.

The battery 261 may include battery cells mounted in a manner to reduce the overall profile of the housing 270. The battery cells may be rechargeable and may be replaceable by opening the housing 270.

The electronics module 250 also includes one or more sensors 280 for measuring parameters of a patient's physiological symptoms. The input/output 266 may include an output module, such as a display or speaker to output notifications or alerts to a patient or caretaker, and/or an input module, such as an input button or other device to receive input from a user.

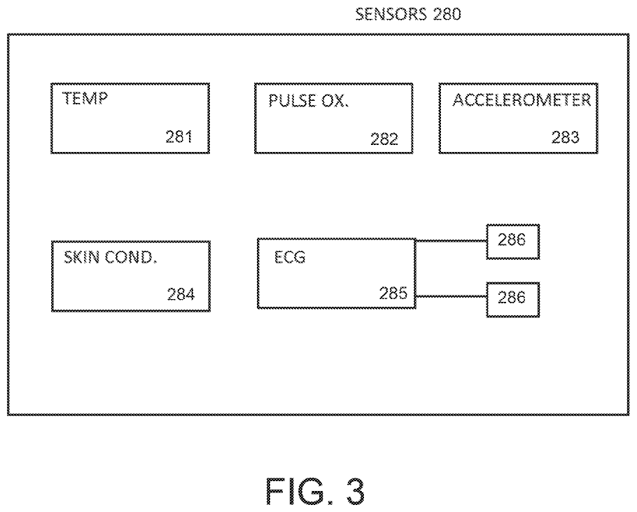

FIG. 3 is a schematic illustration of the sensors 280 of the electronics module 250 according to an example embodiment. A temperature sensor 281 may measure a patient's temperature. An unusually low temperature may be an indicator of hypoglycemia, while a fever may be an indicator of hyperglycemia. An electrocardiogram (ECG) monitor 285 measures the electrical activity of the heart by voltage over time and includes electrodes 286 disposed on a patient's skin. The electrodes 286 detect small electrical changes that are a consequence of cardiac muscle depolarization followed by repolarization during each cardiac cycle. Changes in a normal ECG pattern occur in numerous cardiac abnormalities, and changes in ECG profiles may be an indicator of hypoglycemia. A sensor comprising an ECG monitor may detect a patients ECG pattern via electrodes disposed on the patient's skin. Pulse oximetry is a non-invasive method for measuring a person's oxygen saturation, and peripheral oxygen saturation readings are typically within 2% accuracy of more invasive readings of arterial oxygen saturation. A transmissive pulse oximeter is placed on a thin part of a patient's body, such as an earlobe, and the device passes two wavelengths of light through the body to a photodetector to measure the changing absorbance at each of the wavelengths, allowing it to determine the absorbencies due to the pulsing arterial blood along, excluding venous blood, skin, bone, muscle, and fat. A reflective pulse oximeter does not requires a thin section of a patient's body. A sensor including a pulse oximeter 282 may detect heart rate variability changes which may be an indicator of hypoglycemia or a higher than normal pulse which may be an indicator of hyperglycemia. An accelerometer 283 may be used to derive a patient's respiration rate by measuring inclination and angular changes during breathing. A sensor including an accelerometer may detect changes in breathing which may indicate hypoglycemia or a shortness of breath which may indicate hyperglycemia. A skin conductance sensor 284 may measure a patient's skin's ability to conduct electricity by applying a tiny electric voltage through two electrodes. Accordingly, a sensor including a skin conductance unit may detect excessive sweating which may be an indicator of hypoglycemia or excessively dry skin, which may be an indicator of hyperglycemia.

Accordingly, the electronics module 250 of this example embodiment may include one or more of a temperature sensor 281, an ECG monitor 285, a pulse oximeter 282, an accelerometer 283, and a skin conductance sensor 284.

Each of the sensors 280 of the module 250 are connected to the microcontroller 260 and output signals to the microcontroller 260.

The module may also include a Bluetooth unit 268, connected to the microcontroller 260, to enable a patient to connect the module to push notifications to a user or connect to a diabetes care application or other application (app) on a user's phone or other device. The app could then link the patient or caregiver to the information from the module.

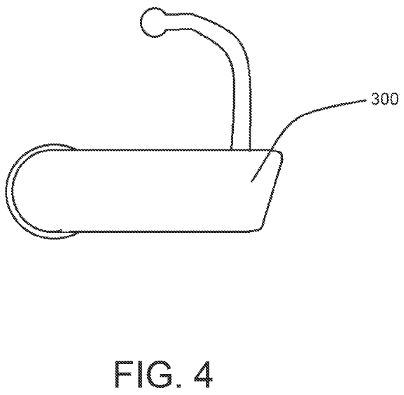

FIG. 4 illustrates an in-ear wearable unit 300 according to another example embodiment. A system including the in-ear wearable unit 300 is for measuring the one or more physiological symptoms via detection within and behind a patient's ear, as shown in FIG. 4.

As with the wrist-wearable unit 200 described above, the in-ear wearable unit 300 comprises an electronics module and may further include an actuator configured to enable the patient to activate or deactivate the physiological monitoring of the electronics module.

The electronics module of the in-ear wearable unit 300 may include all of the elements described above with respect to the electronics module 250 of the wrist-wearable unit 200, including a housing, a microcontroller, a battery, and an actuator. Like the microcontroller 260 of the wrist-wearable module 200, the microcontroller of the in-ear wearable module 300 may be a CMOS integrated circuit comprising the functional elements of a central processing unit (CPU), a read-only memory (ROM), a random access memory (RAM), an electrically-erasable programmable memory (EEPM), a timer, one or more input/output ports, and a programmable voltage reference (VREF), as described above and as shown in FIG. 2.

The electronics module of the in-ear wearable unit 300 may include sensors such as those described above with respect to the wrist-wearable unit 200. Notably, the temperature sensor of the in-ear wearable unit 300 may measure a patient's temperature via detection in the patient's ear.

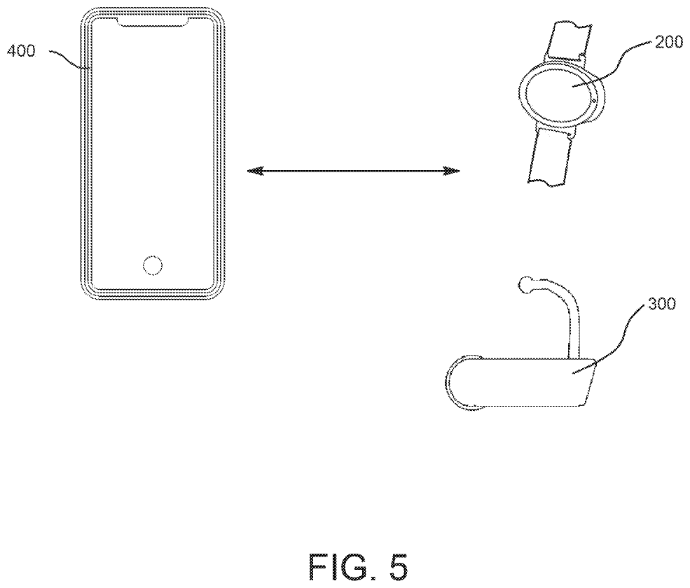

FIG. 5 illustrates a system including one or more of the and wrist-wearable unit 200 and the in-ear wearable unit 300, as well as an external device 400 enabled with an app. The external device 400 may be, for example, a smart phone, as illustrated, or a laptop, tablet, personal computer, or other processing device enabled with an app. The wearable unit 200 or 300 and the external device 400 enabled with the app may be connected wirelessly, by NFC, for example. The two communicating platforms may have different combinations of hardware and software. Data transfer between the devices may differ depending on when and how data transfer occurs between the wearable unit 200 or 300 and the external device 400. The communication connectivity may be conducted via any type of wireless connectivity methods including, but not limited to NFC, Bluetooth™, and WiFi, which may impact device pairing, if needed, and a need for proximity of the devices. The appropriate proximity of the devices relative to each other depends on the connectivity method used, as would be understood by one of skill in the art. The timing of data transfer may depend at least in part on whether or not the two communicating platforms and or at least the wearable unit 200 or 300 has a time recording capability.

In accordance with an aspect of an example embodiment, the external device 400 may be a smart phone provided with a delivery informatics app to connect to and cooperate with the wearable unit 200 or 300. A user may pair the smart phone with the smart app for synchronization using, for example, standard NFC technology methods.

Data synchronization between the wearable unit 200 or 300 and the app can occur at predetermined time intervals, for example. The app may advantageously provide time recording capability (e.g., data provided during synchronization may be stored in the external device 400 or in an external memory, e.g. the cloud, with a time stamp).

Regarding the app described herein, it may be a standalone app stored and operating on a smartphone or other external device 400, as discussed above, or may be provided as an enhancement to a digital health app. The medical event image capture app can also be integrated into a digital health app (e.g., the BD® Diabetes Care app). For example, the app and its generated informatics can be automatically combined with other digital health app content, such as logs of injections, exercise, carbohydrates intake and blood glucose readings, to assist the patient and disease management stakeholders in tracking a patient's compliance with a prescribed disease management regime (e.g., how well the patient is maintaining target blood glucose levels), reordering supplies (e.g., home health supplies such as self-injection devices and medication, and pharmacy inventory) and auto-shipping of prescribed medications and medical supplies to patients or commercial settings, inventory tracking, billing for medical events captured within clinical settings, and the like. Alternately, the app can be a standalone app that communicates with the user (e.g., patient) or other stakeholders on the patient's medical condition management team such as caregivers (e.g., parents, spouses), health care providers, clinical setting administrators, pharmacies, payers (e.g., insurance companies), and medical device suppliers and distributors.

The components of the illustrative devices, systems and methods employed in accordance with the illustrated embodiments described herein can be implemented, at least in part, in digital electronic circuitry, analog electronic circuitry, or in computer hardware, firmware, software, or a combination thereof. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or other device or on multiple device at one site or distributed across multiple sites and interconnected by a communication network. Also, functional programs, codes, and code segments for accomplishing features described herein can be easily developed by programmers skilled in the art. Method steps associated with the example embodiments can be performed by one or more programmable processors executing a computer program, code or instructions to perform functions (e.g., by operating on input data and/or generating an output). Method steps can also be performed by, and apparatuses described herein can be implemented as, special purpose logic circuitry, e.g., a field programmable gate array (FPGA) or an application-specific integrated circuit (ASIC), for example.

The various illustrative logical blocks, modules, and circuits described in connection with the embodiments described herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory (ROM) (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Computer-readable non-transitory media includes all types of computer readable media, including magnetic storage media, optical storage media, flash media and solid state storage media. It should be understood that software can be installed in and sold with a central processing unit (CPU) device. Alternately, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

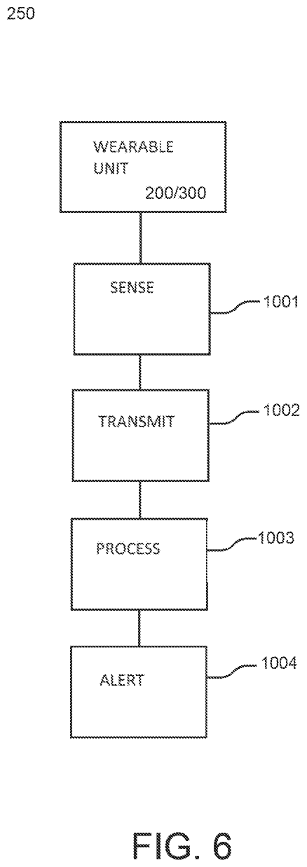

FIG. 6 is a flow chart of operations of the wearable unit 200 or 300 according to an example embodiment. The sensors 280 of the wearable unit 200 or 300 sense physiological symptoms of the patient (1001). Information from the sensors 280 is transmitted to the microcontroller 260 (1002), and the microcontroller 260 processes the received information and determines hypoglycemic or hyperglycemic state of the patient (1003). If the hypoglycemic or hyperglycemic state of the patient is determined to require action, the microcontroller 260 controls the output unit 266 of the wearable unit 200 or 300 to output an alert to the patient (1004). The output unit 2660 may include one or more of an indication light, such as an LED, a vibration unit, and a speaker, and the alert may be more or more of a steady or flashing light, a vibration, and an audible alert such as an alarm or beep, as would be understood by one of skill in the art.

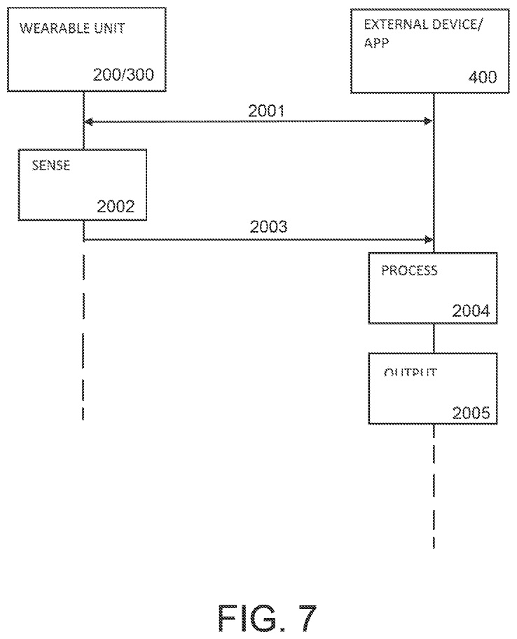

FIG. 7 is a flow chart of operations of a wearable unit 200 or 300 and external device according to an example embodiment. As shown the wearable unit 200 or 300 establishes a connection with the external device 400, establishing communication therebetween (2001). As shown in FIG. 7. The wearable unit 200 or 300 is in operation and senses physiological symptoms of the patient (2002). During operation, information regarding the physiological symptoms or information regarding the hypoglycemic or hyperglycemic state of the patient, as determined by the wearable unit 200 or 300 is transmitted to the app as operated on the external device 400 (2003). The app then processes the received information (2004) and outputs information to the patient (2005).

It may be understood that the example embodiments described herein may be considered in a descriptive sense only and not for purposes of limitation. Descriptions of features or aspects within each example embodiment may be considered as available for other similar features or aspects in other example embodiments.

While example embodiments have been described with reference to the figures, it will be understood by those of ordinary skill in the art that various changes in form and details may be made therein without departing from the spirit and scope as defined by the following claims.

1. A wearable unit comprising:
a housing configured to be worn on a body of a patient; and
an electronics module comprising:
a power source,
plurality of sensors comprising a temperature sensor configured to sense a temperature of the patient, a pulse oximeter configured to sense an oxygen saturation of the patient, an accelerometer configured to sense a breathing pattern of the patient, a skin conductance sensor configured to sense a skin conductance of the patient, and an electrocardiogram (ECG) monitor configured to sense an ECG pattern of the patient, and
a microcontroller configured to receive an output from one or more of the plurality of sensors and to determine, whether the output indicates one of hyperglycemia and hypoglycemia.

2. The wearable unit according to claim 1, further comprising:
a wristband configured to attach the housing to a wrist of the patient.

3. The wearable unit according to claim 2, the microcontroller further comprising a Bluetooth module, wherein the microcontroller is further configured to transmit information to an external device, the information comprising at least one of the output from the one or more of the plurality of sensors and a determination of whether the output indicates one of hyperglycemia and hypoglycemia.

4. The wearable unit according to claim 1, further comprising:
an earpiece configured to hold the housing within an ear of the patient.

5. The wearable unit according to claim 4, the microcontroller further comprising a Bluetooth module, wherein the microcontroller is further configured to transmit information to an external device, the information comprising at least one of the output from the one or more of the plurality of sensors and a determination of whether the output indicates one of hyperglycemia and hypoglycemia.

5. (canceled)

6.-15. (canceled)

16. The wearable unit according to claim 1, the electronics module further comprising:
an output unit,
wherein the microcontroller is further configured to output at least one of a visible alert and an audible alert to the patient based on whether the output indicates one of hyperglycemia and hypoglycemia.

17. A sensing system comprising:
an external device; and
a wearable unit comprising:
a housing configured to be worn on a body of a patient; and
an electronics module comprising:
a power source,
plurality of sensors comprising a temperature sensor configured to sense a temperature of the patient, a pulse oximeter configured to sense an oxygen saturation of the patient, an accelerometer configured to sense a breathing pattern of the patient, a skin conductance sensor configured to sense a skin conductance of the patient, and an electrocardiogram (ECG) monitor configured to sense an ECG pattern of the patient,
a transmission unit; and
a microcontroller configured to:
receive an output from one or more of the plurality of sensors, and
control the transmission unit to output, to the external device, information comprising the output from the one or more of the plurality of sensors;
wherein the external device is configured to receive the information from the wearable device and to determine whether the output indicates one of hyperglycemia and hypoglycemia.

18. The sensing system according to claim 17, the wearable unit further comprising a wristband configured to attach the housing to a wrist of the patient.

19. The sensing system according to claim 17, wherein the transmission unit comprises a Bluetooth module.

20. The sensing system according to claim 17, the wearable unit further comprising an earpiece configured to hold the housing within an ear of the patient.

21. The sensing system according to claim 20, wherein the transmission unit comprises a Bluetooth module.

22. The sensing system according to claim 17, wherein the external device is further configured to determine to output at least one of a visible alert and an audible alert to the patient based on whether the output indicates one of hyperglycemia and hypoglycemia.

23. A sensing method comprising:
a wearable unit sensing a temperature of a patient wearing the wearable unit, an oxygen saturation of the patient, a breathing pattern of the patient, a skin conductance of the patient, and an electrocardiogram (ECG) pattern of the patient;
transmitting, from the wearable unit to an external device, the oxygen saturation, the breathing pattern, the skin conductance, and the electrocardiogram (ECG) pattern;
the external device determining whether the output indicates one of hyperglycemia and hypoglycemia; and
the external device outputting at least one of a visible alert and an audible alert based on whether the output indicates one of hyperglycemia and hypoglycemia.

24. The sensing method according to claim 23, wherein the transmitting comprises transmitting via a Bluetooth module.

25. The sensing method according to claim 23, wherein the wearable unit is configured to be attached to a wrist of the patient.

26. The sensing method according to claim 23, wherein the wearable unit is configured to be worn in an ear of the patient.
